(19) United States
(12) Patent Application Publication
(10) Pub. No.: US 2026/0286271 A1
(43) Pub. Date: Sep. 24, 2026

(54) DEVICES, SYSTEMS AND METHODS FOR ASEPTICALLY SAMPLING CELL CULTURES

(71) Applicant: Kite Pharma, Inc., Santa Monica, CA (US)

(72) Inventors: Oliver D. Lin, Foster City, CA (US); Dmitry Fradkin, Santa Monica, CA (US)

(21) Appl. No.: 19/563,168

(22) Filed: Mar. 11, 2026

Related U.S. Application Data
(60) Provisional application No. 63/770,881, filed on Mar. 12, 2025.

(51) Int. Cl. C12M 1/26; C12M 1/34; G01N 15/10; G01N 15/14

(52) U.S. Cl. CPC C12M 33/14; C12M 41/00; G01N 15/14; G01N 2015/1006; G01N 2015/1486

(57) ABSTRACT

The present disclosure describes technology for sampling a fluid from a cell culture system or device. In various embodiments, the described methods may be performed by the described system in an automated or semi-automated fashion, thereby, reducing time and expense. In accordance with the described technology, samples may then be analyzed.

### CROSS-REFERENCE TO RELATED APPLICATIONS

This application claims the benefit under 35 U.S.C. § 119(e) of U.S. Provisional Application No. 63/770,881 filed on Mar. 12, 2025 which is hereby incorporated herein by reference in its entirety for all purposes.

### FIELD OF THE INVENTION

The present disclosure generally relates to systems and methods for sampling lots undergoing a cell culture. More specifically, the disclosure relates to systems and methods for aseptically drawings samples from a cell culture to keep the cell culture and/or the samples sterile.

### BACKGROUND

As a part of design and quality control of cell therapy products, there is a need to sample cell therapy products at various stages of production. For example, when engineered T cells (e.g., CAR T cells) go through a manufacturing process it may be important to assess cell viability, total cell count, purity, specific gene expression (e.g., CAR expression), functionality, and take measurements for possible contamination. In various applications, drawing a sample from a larger lot may be helpful in making these assessments.

Current sampling systems are both expensive and time consuming. For example, many systems and methods rely on heavy use of highly skilled technical people to prepare systems and/or carry out the methods manually. Another problem with methods being carried out manually is lack of repeatability. Additionally, systems often require complex cleaning protocols to ensure contaminants are reduced or eliminated and/or sterility is maintained in both the larger lot and/or the sample being drawn from the lot.

The systems and methods described herein solve these and additional problems by reducing labor costs and increasing repeatability while increasing the likelihood of reducing or eliminating contamination and/or maintaining sterility of a cell culture.

### SUMMARY

In various aspects, a system for sampling a cell culture fluid is described herein. In various embodiments, a fluidic assembly may comprise a first fluid channel having a first end and a second end, a second fluid channel having a first end and a second end, a third fluid channel having a first end and a second end, and a sample chamber having a predefined volume. In various embodiments, the sample chamber may be fluidically coupled to the second end of the first fluid channel at a first position, the second end of the second fluid channel at a second position, and the second end of the third fluid channel at a third position. In various embodiments, the fluidic assembly may be mounted to a fluidic handling system. In various embodiments, the fluidic handling system may comprise a first valve in physical communication between the first and second ends of the first fluid channel and a second valve in physical communication between the first and second ends of the second fluid channel.

In various embodiments, the fluidic assembly may further comprise a check valve positioned between the first end and the second end of the second fluid channel and a filter affixed to the first end of the third fluid channel.

In various embodiments, the fluidic handling system may further comprise a first sensor in physical, sonic, or optical communication at a position between the first and the second ends of the first fluid channel, wherein the first sensor is for detecting the presence of a liquid and a second sensor in physical, sonic, or optical communication at a position between the first and the second ends of the third fluid channel, wherein the second sensor is for detecting the presence of a liquid.

In various embodiments, the system for sampling the cell culture fluid may further comprise a fourth channel having a first end and a second end. In various embodiments, the filter may be affixed to the second end of the fourth channel and a pressure sensor located between the first and seconds ends of the fourth channel and in fluidic communication with the fourth channel. In various embodiments, the filter may comprise a luer lock for securing the second end of the fourth fluid channel to the first end of the third fluid channel. In various embodiments, the system for sampling the cell culture fluid may further comprise a connector channel. In various embodiments, the connector channel may fluidically connect the pressure sensor to the fourth fluid channel.

In various embodiments, the fluid channels may be each comprised of a flexible tube surrounding a hollow interior.

In various embodiments, the first and second valves may each comprise a pinch valve.

In various embodiments, the system for sampling the cell culture fluid may further comprise a pump fluidically coupled to the first end of the fourth channel. In various embodiments, the pump may be selected from any one of a syringe pump, a diaphragm pump, a peristaltic pump, or any pump capable of moving and compressing air.

In various embodiments, the system for sampling the cell culture fluid may further comprise a cell culture vessel fluidically coupled to the first end of the first fluid channel. In various embodiments, the system for sampling the cell culture fluid may further comprise a collection device fluidically coupled to the first end of the second fluid channel.

In various embodiments, the system for sampling the cell culture fluid may further comprise a collection device sensor for physically, sonically or optically interacting with a fluid inside of the collection device. In various embodiments, the collection device comprises an analytical device. In various embodiments, the analytical device may be selected from any one of a cell counter, a flow cytometer, a cell imaging system.

In various aspects, a collection device sensor for detecting the presence of the collection device is provided. In various embodiments, the collection device sensor comprises a light emitting element, wherein the light emitting element operates at a first energy level to detect the presence of the collection device and a second energy level to interrogate a fluid within the collection device.

In various aspects, a sensor for interrogating a fluid within the collection device is provided. In various embodiments, the sensor physically, sonically, or optically interacts with the fluid.

In various embodiments, the system for sampling the cell culture fluid may further comprise a controller. In various embodiments, the controller may be in electronic communication with the pump, the pressure sensor, the first and second sensors, and the first and second values. In various embodiments, the system for sampling the cell culture fluid may further comprise a human machine interface in electronic communication with the control unit.

In various embodiments, the controller may further comprise a micro-controller, a valve driver, a memory, and a pump driver. In various embodiments, the human machine interface may further comprise a display and controls.

In various aspects, a method for sampling a cell culture fluid is described. In various embodiments, the method for sampling a cell culture fluid may comprise applying a negative pressure to draw a cell culture sample from a cell culture vessel into a sample chamber through a first channel until the cell culture sample occupies a predefined volume of the sample chamber, actuating a first valve to a closed position to prevent a backflow of the cell culture sample from the sample chamber into the cell culture vessel, actuating a second valve to an open position to allow access of the cell culture sample from the sample chamber to a collection device through a second channel, and applying a positive pressure to push the cell culture sample from the sample chamber to the collection device through the second channel.

In various embodiments, the method for sampling a cell culture fluid may further activating a pump in a forward direction to apply the positive pressure and activating the pump in a reverse direction to apply the negative pressure.

In various embodiments, the method for sampling a cell culture fluid may comprise detecting the presence of the cell culture sample within the first channel using a first sensor.

In various embodiments, the method for sampling a cell culture fluid may comprise detecting the presence of the cell culture sample using a second sensor and discontinuing the application of negative pressure once detection of the presence of the cell culture by the second sensor has occurred.

In various embodiments, the second sensor may detect the cell culture sample in the sample chamber. In various embodiments, the second sensor may detect the cell culture sample in a third channel, wherein the third channel is in fluid communication with the sample chamber.

In various embodiments, the method for sampling a cell culture fluid may comprise preventing the cell culture fluid from entering a fourth channel from the third channel using a filter. In various embodiments, the fourth channel connects the pump to the third channel.

In various embodiments, the method for sampling a cell culture fluid may comprise preventing backflow from the collection device to the sample chamber using a check valve. In various embodiments, the check valve may be positioned along the second channel.

In various embodiments, the method for sampling a cell culture fluid may comprise mounting a fluidic assembly to a fluidic handling system. In various embodiments, the fluidic assembly may comprise the first, second, and third channels, the filter, and the check valve. In various embodiments, the fluidic handling system comprises the first and second sensors, the first and second valves, and the pump.

In various embodiments, the mounting step may comprise welding a first end of the first channel to the cell culture vessel.

In various embodiments, the method for sampling a cell culture fluid may comprise performing an integrity test of the fluidic assembly by activating the pump to apply pressurized air to the fluidic assembly and checking for leaks prior to drawing the cell culture sample.

In various embodiments, the method for sampling a cell culture fluid may comprise collecting data from the first and second sensors using a control system, actuating the first and second valves based on the data received by the control system, and activating the pump in one of a forward or reverse direction based on the data and the position of the first and second valves.

In various embodiments, the method for sampling a cell culture fluid may comprise analyzing the cell culture sample for a quality. In various embodiments, the quality includes a cell count. In various embodiments, the quality includes a contaminant. In various embodiments, the quality includes cell viability information for one or more populations of cells within the cell culture sample. In various embodiments, the quality includes morphology information for one or more populations of cells within the cell culture sample. In various embodiments, the quality includes protein expression information for one or more populations of cells within the cell culture sample. In various embodiments, protein expression may include cell surface protein expression.

### BRIEF DESCRIPTION OF THE FIGURES

FIG. 1 is a schematic diagram of a system for aseptically sampling a fluid in accordance with various embodiments.

FIG. 2 is a schematic diagram of a fluidic assembly in accordance with various embodiments.

FIG. 3 is a schematic diagram of a fluidic handling system in accordance with various embodiments.

FIG. 4 is a schematic diagram of a control system for a system for aseptically sampling a fluid in accordance with various embodiments.

FIG. 5 is a schematic diagram of a computer system in accordance with various embodiments.

FIG. 6 is a method for aseptically sampling a fluid in accordance with various embodiments.

### DETAILED DESCRIPTION OF CERTAIN EMBODIMENTS

The present disclosure, among other things, provides insights and technologies useful for sampling a fluid from a cell culture system or device. In various embodiments, the technology comprises systems and methods for keeping the cell culture sterile. In various embodiments, the technology comprises systems and methods for keeping the sample sterile. In various embodiments, the technology comprises systems and methods for keeping both the cell culture and the sample sterile. Further, the embodiments described herein may be useful in the life sciences and pharmaceutical industries to ensure patient safety and regulatory compliance. For example, detecting, characterizing, and/or identifying a characteristic of a beginning, intermediate, and/or final product (e.g., a cell therapy product for a patient) may provide information useful in determining whether a product is suitable for administering to a patient.

Embodiments of systems, apparatuses, and methods for aseptically sampling a fluid from a cell culture system or device are described in the accompanying description and figures. In the figures, numerous specific details are set forth to provide a thorough understanding of certain embodiments. A skilled artisan will appreciate that the systems and methods described herein may be used in a variety of ways and circumstances that are not limited to what is specifically detailed. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences can be varied and still remain within the spirit and scope of certain embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

### Exemplary Systems

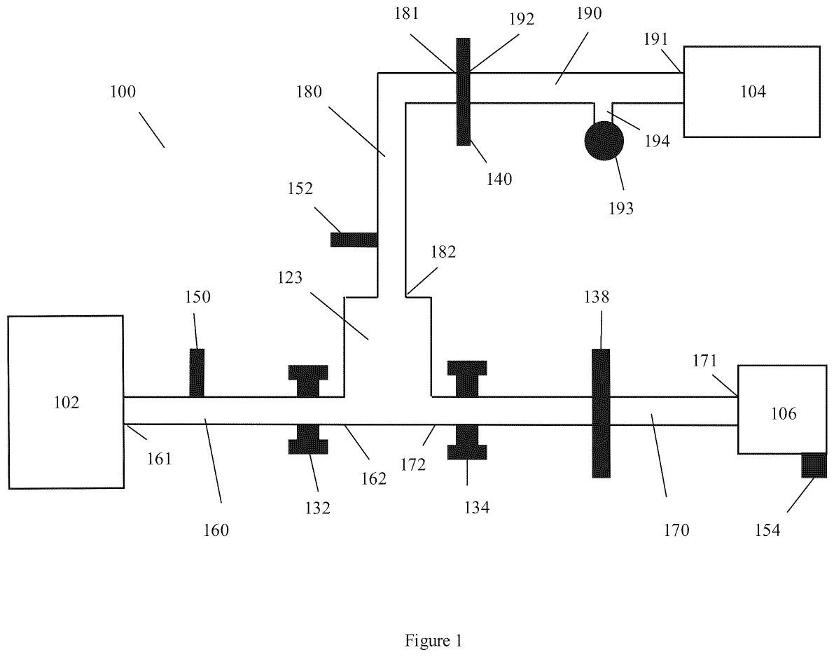

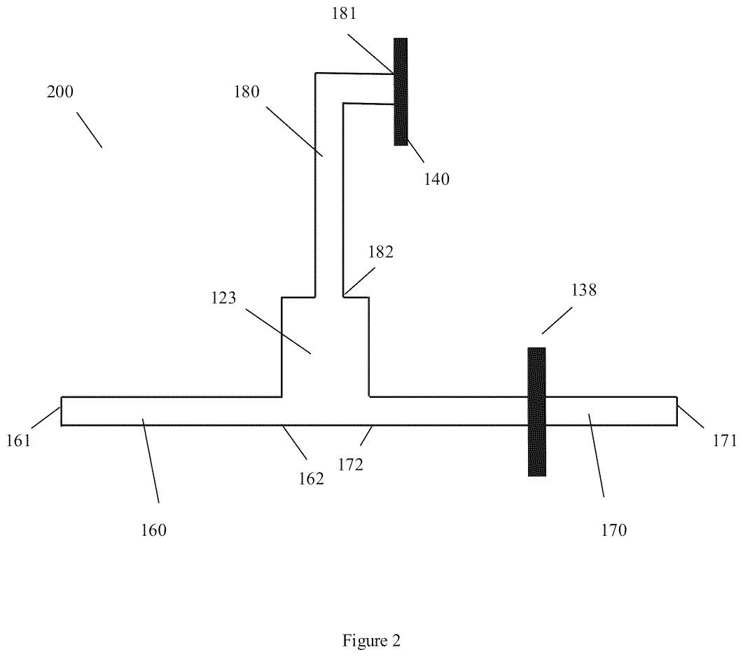

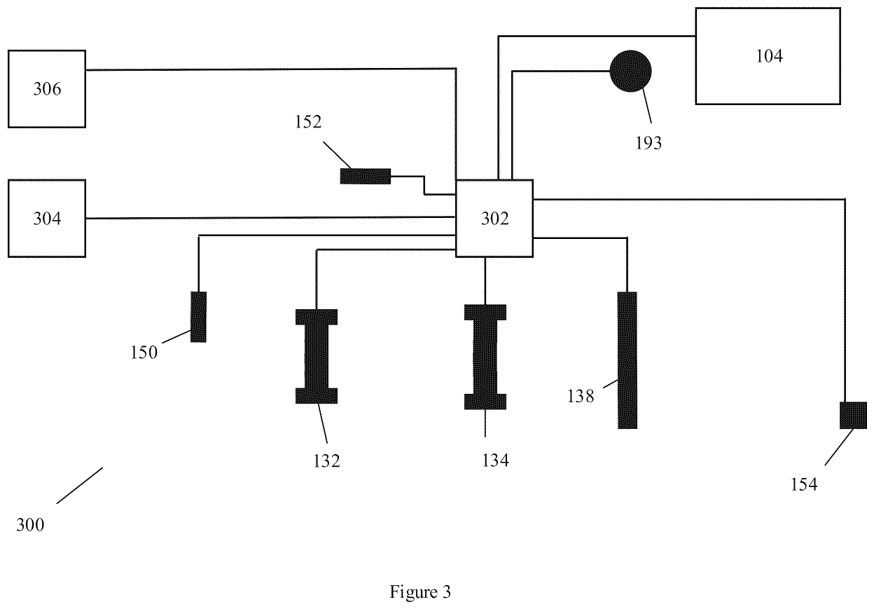

FIG. 1 is a schematic diagram of a system for aseptically sampling a fluid 100 in accordance with various embodiments. In various embodiments, the system for aseptically sampling a fluid 100 may comprise a fluidic assembly 200 (see FIGS. 1 and 2) and a fluidic handling system 300 (see FIGS. 1 and 3).

FIG. 2 is a schematic diagram of a fluidic assembly 200 in accordance with various embodiments. In various embodiments, a fluidic assembly 200 may comprise a first fluid channel 160 having a first end 161 and a second end 162. In various embodiments, a fluidic assembly 200 may comprise a second fluid channel 170 having a first end 171 and a second end 172. In various embodiments, a fluidic assembly 200 may comprise a third fluid channel 180 having a first end 181 and a second end 182.

In various embodiments, a fluidic assembly 200 may comprise a sample chamber 123. In various embodiments, the sample chamber 123 may have a predefined volume. In various embodiments, the predefined volume may comprise 1 mL. In various embodiments, the predefined volume may comprise between about 0.5 mL to about 1.5 mL. In various embodiments, the predefined volume may comprise between about 0.1 mL to about 1.5 mL. In various embodiments, the predefined volume may comprise up to 1 mL. In various embodiments, the predefined volume may comprise up to 1.5 mL. In various embodiments, the predefined volume may comprise up to 2 mL. In various embodiments, the predefined volume may comprise down to 1 mL. In various embodiments, the predefined volume may comprise down to 900 uL. In various embodiments, the predefined volume may comprise down to 800 uL. In various embodiments, the predefined volume may comprise down to 700 uL. In various embodiments, the predefined volume may comprise down to 600 uL. In various embodiments, the predefined volume may comprise down to 500 uL. In various embodiments, the predefined volume may comprise down to 400 uL. In various embodiments, the predefined volume may comprise down to 300 uL. In various embodiments, the predefined volume may comprise down to 200 uL. In various embodiments, the predefined volume may comprise down to 100 uL.

However, the skilled artisan will appreciate that depending on the intended assays for the sample any size predefined volume may be engineered. For example, flow cytometry assays may comprise a running sample volume so the predefined sample may be customized to one or more running sample volumes for one or more analytical instruments.

In various embodiments, the sample chamber 123 may be fluidically coupled to the second end 162 of the first fluid channel 160 at a first position of the sample chamber 123. In various embodiments, the sample chamber 123 may be fluidically coupled to the second end 172 of the second fluid channel 170 at a second position of the sample chamber 123. In various embodiments, the sample chamber 123 may be fluidically coupled to the second end 182 of the third fluid channel at a third position of the sample chamber 123.

In various embodiments, the sample chamber 123 may comprise a rectangular shape. In various embodiments, the first position and the second position may be located on opposing surfaces of the sample chamber 123. In various embodiments, the first, second, and third positions may be located on different walls of a predefined shape of the sample chamber 123 (e.g., shapes having three or more sides). In various embodiments, the first and second positions of the sample chamber 123 may be located at a based or bottom position of the sample chamber 123 and be opposed to one another while the third position may be located perpendicular (e.g., a top or upper location) to the first and second positions.

In various embodiments, the sample chamber 123 may comprise a triangle shape. In such embodiments, the first, second, and third positions may be located at the points of the triangle. For example, the first and second positions may oppose one another at a lower, or bottom positions and the third position may be perpendicular to the first and second positions. In various embodiments, the first, second, and third positions may be located at or near the walls of the triangle.

In various embodiments, the sample chamber 123 may comprise a cylindrical shape. In such embodiments, the first, second, and third positions may be located along a sidewall or at a top or bottom surface and in any orientation conceivable.

In various embodiments, a sample chamber 123 may be shaped in a manner that when installed on a fluidic handling system, opening 182 (connection to filter/pump) may be directly vertical above openings 162 & 172. In such embodiments, this may ensure the sample chamber 123 may be filled fully without premature overflow into the third channel 180.

In various embodiments, sample chamber 123 size may depend on the requirements of the sampling operation. In various embodiments, the sample chamber 123 may comprise a volume of 1 mL. In various embodiments, the sample chamber 123 may comprise a volume within 90% of 1 mL. In various embodiments, the sample chamber 123 may comprise a volume within 80% of 1 mL. In various embodiments, the sample chamber 123 may comprise a volume within 70% of 1 mL. In various embodiments, the sample chamber 123 may comprise a volume within 60% of 1 mL. In various embodiments, the sample chamber 123 may comprise a volume within 50% of 1 mL. In various embodiments, the sample chamber 123 may comprise a volume within 40% of 1 mL. In various embodiments, the sample chamber 123 may comprise a volume within 30% of 1 mL. In various embodiments, the sample chamber 123 may comprise a volume within 20% of 1 mL. In various embodiments, the sample chamber 123 may comprise a volume within 10% of 1 mL.

In various embodiments, a cylindrically shaped sample chamber 123 may be suitable for connecting to the channels 160, 170, 180. In various embodiments, any other shape may be suitable. A consideration in sample chamber 123 design may include selection of shape based on design of other components, in accordance with various embodiments. A consideration in sample chamber 123 design may include selection of shape or volume to reduce liquid retention in the system after sample collection is completed.

In various embodiments, channels 160, 170, 180 may be connected to the sample chamber 123 or any other component of the system 100 using any fluidic connection, including but not limited to, barbed fittings, ultrasonic or heat welding, and/or glue.

In various embodiments, the fluidic assembly 200 may comprise a check valve 138. In various embodiments, the check valve 138 may be positioned between the first end 171 and the second end 172 of the second fluid channel 170.

In various embodiments, a check valve 138 may prevent any meaningful backflow of both liquids and gas to ensure contamination does not reach the sterile portion of the fluidic assembly 200. In various embodiments, the check valve 138 may allow passage of liquids and gases in an opposing direction with minimal fluidic resistance. Acceptable types may include, but are not limited to, diaphragm check valves, duckbill check valves, ball check valves, etc.

In various embodiments, the fluidic assembly 200 may comprise a filter 140. In various embodiments, the filter 140 may be positioned or affixed to the first end 181 of the third fluid channel 180. In various embodiments, the filter 140 may be designed to prevent passage of a liquid out of the third fluid channel 180 while allowing passage of a gas.

In various embodiments, a filter 140 may be designed or selected to prevent microbial ingress while also permitting gas flow.

In various embodiments, the filter 140 may be connected to the first end 181 of the third fluid channel 180 by weld or by an aseptic connector. In various embodiments, the filter 140 may comprise a luer lock securing a second end 192 of a fourth fluid channel 190 to the first end 181 of the third fluid channel 180 (see FIG. 1).

In various embodiments, connection 192 from filter 140 to pump 104 tubing may be designed to be removable (i.e. to allow replacement of consumable). In various embodiments, connection 192 may comprise a luer lock. In various embodiments, a luer lock may be preferable but any airtight removable connection may be considered. In various embodiments, connection 192 may include twist-to-connect and/or push-to-connect fittings.

In various embodiments, filter 140 may be used to prevent microbial ingress from non-sterile sections 190. In various embodiments, filter 140 may comprise a hydrophobic membrane to help prevent liquid ingress into pump if the liquid sensor 152 does not respond. In various embodiments, filter 140 may comprise pores having a size of 0.2 um or smaller. In various embodiments, 0.2 um may be adequate for microbial protection. However, in various embodiments, other pore sizes may be selected based on characteristics of a contaminant. In various embodiments, a Steridyne™ membrane filter may be suitable.

In various embodiments, the fluid channels 160, 170, 180, 190 may be each comprised of a flexible tube surrounding a hollow interior.

In various embodiments, the fluidic assembly 200 may comprise a fourth channel 190 having a first end 191 and a second end 192. In various embodiments, a filter 140 may be affixed to the second end 192 of the fourth channel 190. In various embodiments, the filter 140 may comprise a luer lock for securing the second end 192 of the fourth fluid channel 190 to the first end 181 of the third fluid channel 180.

In various embodiments, a connector channel 194 may fluidically connect a pressure sensor 193 to the fourth fluid channel 190. In various embodiments, the pressure sensor 193 may serve to determine whether the pump 104 is operating at a desired rate, thereby, applying a desired quantity of pressure to the fluidic assembly 200. In various embodiments, the pressure sensor 193 may detect high pressure during pumping to indicate a blockage somewhere in the system.

In various embodiments, the first and second valves each comprise a pinch valve.

FIG. 3 is a schematic diagram of a fluidic handling system 300 in accordance with various embodiments. In various embodiments, a system for aseptically sampling a fluid 100 may comprise the fluidic handling system 300 (see FIGS. 1 and 3). In various embodiments, a fluidic assembly (see FIGS. 1 and 2) may be mounted to the fluidic handling system 300 (see FIGS. 1 and 3).

In various embodiments, the fluidic handling system 300 may comprise a first valve 132 in physical communication between the first and second 161, 162 ends of the first fluid channel 160. In various embodiments, the fluidic handling system 300 may comprise a second valve 134 in physical communication between the first and second ends 171, 172 of the second fluid channel 170.

In various embodiments, valves may be non-liquid contact valves to avoid contaminating the sample fluid. In various embodiments, a valve may comprise a solenoid-operated pinch valve, which allows fluid flow control by clamping the tubing shut from the exterior.

In various embodiments, the fluidic handling system 300 may comprise a first sensor 150 in physical, sonic, or optical communication at a position between the first and the second ends 161, 162 of the first fluid channel 160. In various embodiments, the first sensor 150 may be for detecting the presence of a liquid. In various embodiments, the first sensor 150 may measure one or more of an optical characteristic, a capacitance, and/or an ultrasonic quality.

In various embodiments, both sensors 150, 152 may have the same requirements. In various embodiments, both sensors 150, 152 may be able to detect a presence and an absence of liquid in the tubing 160, 180.

In various embodiments, sensor 150 may detect initial filling of a consumable. In various embodiments, if the sensor does not detect liquid after preset time, then problem may have occurred, and a warning can be displayed.

Sensor 152 is to detect when the liquid has fully filled chamber 123, after which the pump is stopped. If sensor 152 malfunctions, liquid may reach filter 140 but hydrophobic membrane should prevent liquid from reaching pump. Additionally, liquid saturating the membrane would result in large pressure spike detectable by pressure sensor

In various embodiments, the fluidic handling system 300 may comprise a second sensor 152 in physical, sonic or optical communication at a position between the first and the second ends 181, 182 of the third fluid channel 180. In various embodiments, the second sensor 152 may measure one or more of an optical characteristic, a capacitance, and/or an ultrasonic quality.

In various embodiments, the fluidic handling system 300 may comprise a fourth channel 190 having a first end 191 and a second end 192. In various embodiments, a filter 192 may be affixed to the second end 192 of the fourth channel 190. In various embodiments, the filter 140 may comprise a luer lock for securing the second end 192 of the fourth fluid channel 190 to the first end 181 of the third fluid channel 180.

In various embodiments, the fluidic handling system 300 may comprise a pressure sensor 193 located between the first and seconds ends 191, 192 of the fourth channel 190 and in fluidic communication with the fourth channel 190. In various embodiments, the filter 140 prevents liquid from entering the fourth channel 190. As such, the pressure sensor 193 may detect pressured fluid in the form of a gas according to various embodiments. In various embodiments, the gas may be comprised of ambient, sterile air. In various embodiments, everything as part of the fluidic assembly 200 may be sterile and the channel 190 from the filter 140 to the pump 104 may not be sterile. In various embodiments, the gas may be comprised of nitrogen. In various embodiments, the gas may be comprised of one of more of helium, neon, argon, krypton, xenon, and/or radon.

In various embodiments, the pressure sensor may be diaphragm-based, piezoelectric, or capacitance based.

Referring back to FIG. 1, a system for aseptically sampling a fluid 100 may further comprise a cell culture vessel 102 and a collection device 106 in addition to a fluidic assembly 200 and a fluidic handling system 300.

In various embodiments, a fluidic assembly (see FIGS. 1 and 2) may be mounted to the fluidic handling system 300 (see FIGS. 1 and 3).

In various embodiments, a fluidic assembly 200 may interact with the fluidic handling system 300 at pinch valves 132, 134, liquid sensors 150, 152, and luer lock connection to the filter 140. In various embodiments, one or more of such connections may adequately hold the fluidic assembly 200 in place. However, in various embodiments, one or more of the following elements may be useful to assist in mounting the consumable as well as managing excess tubing: 1) Brackets sized to allow the tubing to be press fit into them and held via friction. 2) Hooks to allow tubing to be held loosely. 3) Straps or ties attached to the hardware to secure the channels in place. 4) In various embodiments, the system may also comprise corresponding labels on both the fluidic assembly 200 and fluidic handling system 300 to assist a user in attaching them together correctly.

In various embodiments, the system for aseptically sampling a fluid 100 may comprise a fourth channel 190 having a first end 191 and a second end 192. In various embodiments, a filter 140 may be affixed to the second end 192 of the fourth channel 190. In various embodiments, the filter 140 may comprise a luer lock for securing the second end 192 of the fourth fluid channel 190 to the first end 181 of the third fluid channel 180.

In various embodiments, the system for aseptically sampling a fluid 100 may comprise a connector channel 194, In various embodiments, the connector channel 194 may fluidically connect the pressure sensor 193 to the fourth fluid channel 190.

In various embodiments, the system for aseptically sampling a fluid 100 may comprise a pump 104 fluidically coupled to the first end 191 of the fourth channel 190. In various embodiments, the pump 104 may be selected from any one of a syringe pump, a diaphragm pump, a peristaltic pump, or any pump capable of moving and compressing air.

In various embodiments, one or more gas pumps may be well suited for the systems described herein. In various embodiments, both positive and negative pressure may be applied. As such, in various embodiments, the pump may be reversible (i.e., may pump in a forward or backward direction). In various embodiments, a mechanism of valves may be used, thereby, allowing the pump to direct flow in either direction.

In various embodiments, a pump 104 may comprise controllable flow rate settings and be capable of metering small volumes of gas. In various embodiments, methods involving metering small volumes of gas may allow for avoidance filter wetting from rapid filling. For example, a flow rate for the pumps 104 described herein an operating rate may comprise about 5 mL/min.

In various embodiments, pump 104 may be able to generate enough pressure to aspirate sample from bioreactor 102 through channels 160, 170 and dispense through the check valve 138.

In various embodiments, a pump 104 does not need to be sterile. However, in various embodiments, the pump 104 may not generate airborne chemical contaminants (e.g. volatile hydrocarbons from oil lubricants) that may be able to travel through the filter 140 and negatively affect cells.

In various embodiments, a pump 104 may comprise a piezoelectric pump, a piston pump, and/or a gear pump.

In various embodiments, the system for aseptically sampling a fluid 100 may comprise a cell culture vessel 102 fluidically coupled to the first end 161 of the first fluid channel 160. In various embodiments, a fluidic assembly 200 may be connected to any bioreactor that has a sampling port or sampling circulation system where a sample can be withdrawn using a pressure gradient. In various embodiments, this may include but is not limited to stirred tank, wave/rocker, fixed-bed, and gas permeable membrane bioreactor types. In various embodiments, connection between bioreactor 102 and a fluidic assembly 200 may preserve sterility using either sterile tube welding or aseptic connectors/couplings (e.g. AseptiQuik™).

In various embodiments, the system for aseptically sampling a fluid 100 may comprise a collection device 106 fluidically coupled to the first end 171 of the second fluid channel 170. In various embodiments, the collection device 106 may comprise a small container or tube with volume sufficient to hold a sample dispensed from the system 100. In various embodiments, the collection device 106 may allow for transport of sample for storage or analytical testing. In various embodiments, a collection device 106 may be low cost and/or disposable. In various embodiments, the collection device 106 may be sterile. In various embodiments, the collection device 106 may be non-sterile.

In various embodiments, the system for aseptically sampling a fluid 100 may comprise a collection device sensor 154 for physically, sonically, or optically interacting with a fluid inside of the collection device 106. In various embodiments, the device sensor 154 may measure one or more of an optical characteristic, a capacitance, and/or an ultrasonic quality. In various embodiments, collection device sensor 154 may measure the presence of a collection device 106. In various embodiments, collection device sensor 154 may measure the presence of a fluid within the collection device 106.

In various embodiments, collection device sensor 154 may be for physical sensing (e.g., a switch triggered by direct physical contact).

In various embodiments, a collection device sensor for detecting the presence of the collection device may be included in the system. In various embodiments, the collection device sensor comprises a light emitting element, wherein the light emitting element operates at a first energy level to detect the presence of the collection device and a second energy level to interrogate a fluid within the collection device. In various embodiments, the system may further comprise a sensor for interrogating a fluid within the collection device. In various embodiments, the sensor physically, sonically, or optically interacts with the fluid.

In various embodiments, the collection device 106 may comprise an analytical device or a component of the analytical device (e.g., a sample cartridge). In various embodiments, the analytical device may be selected from any one of a cell counter, a flow cytometer, a cell imaging system. In various embodiments, the analytical device may comprise a biochemical analyzer.

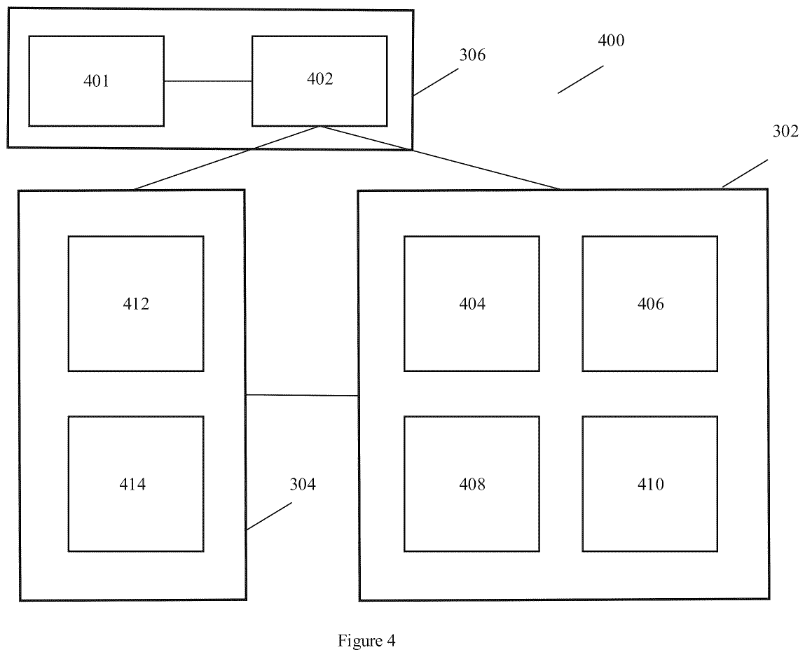

FIG. 4 is a schematic diagram of a control system 400 for a system for aseptically sampling a fluid 100 in accordance with various embodiments.

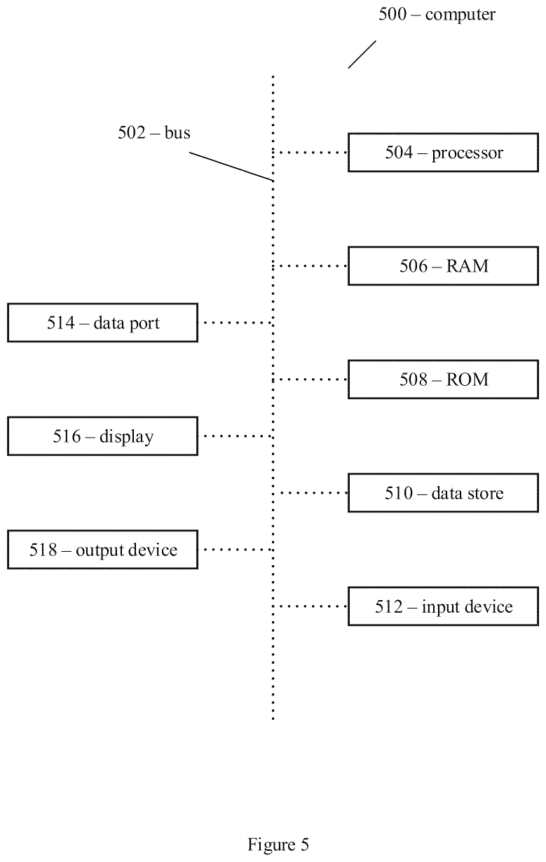

Referring to FIGS. 3 and 4, in various embodiments, the control system 400 may comprise a controller 302 and a human machine interface 304. In various embodiments, the controller 302 may be comprised of one or more of the components described in FIG. 5. In various embodiments, the human machine interface 304 may be comprised of one or more of the components described in FIG. 5.

In various embodiments, the controller 302 may be in electronic communication with the pump 104, the pressure sensor 193, the first and second sensors 150, 152, and the first and second values 132, 134. In various embodiments, the controller 302 may be in electronic communication with a human machine interface 304.

In various embodiments, electronic communication may comprise wired, wireless, or any known or useful device of lack thereof for electrically motivated materials to communicate information.

In various embodiments, the controller 302 may further comprise one or more of a micro-controller 404, a valve driver 406, a memory 408, and a pump driver 410.

In various embodiments, the human machine interface 304 may further comprise a display 412 and controls 414. In various embodiments, the human machine interface 304 may comprise or be comprised of one or more of the components described in FIG. 5.

In various embodiments, the control system 400 may comprise a power supply 306. In various embodiments, the power supply 306 may comprise a power source 401 and a power converter 402. In various embodiments, the power source 401 may be a battery or a hardwire to an electrical grid. In various embodiments, the power converter 402 may be responsible for converting alternating current to direct current.

In various embodiments, the HMI 304 may be connected to the micro-controller 404, and an operator may interact with the HMI 304 to start an auto-sampling routine. One or more auto-sampling programs/routines may be stored in non-volatile memory and run via the micro-controller 404.

In various embodiments, a routine may open valve 132 allowing a sample to be drawn from a cell culture 102. Valves 132, 134 may be actuated using a valve driver 406, which may convert a low power digital signal from the micro-controller 404 to a high power to open/close the solenoid pinch valves 132, 134.

In various embodiments, a micro-controller 404 may start a pump 104 to draw sample into a chamber 123 at a pre-defined flow rate. The micro-controller 404 may initiate the pump 104 operation through a pump driver 410, which may convert the digital signal from the micro-controller 404 into electrical current that can power a pump 104. The specific details of the pump driver 410 vary depending on the type of pump used.

Depending on the sensor, the micro-controller 404 may receive analog (voltage level) or digital signals from the sensors and may convert the analog signal into a digital reading via onboard I/O.

The auto-sampling program may check the value of liquid sensor 150 until it detects liquid. In various embodiments, if it does not detect liquid within a preset amount of time, there may a sampling error. In various embodiments, an error message may be displayed on the HMI display 412. If the liquid sensor 150 is triggered correctly, the program may then continue filling until liquid sensor 152 detects liquid, at which point the micro-controller 404 may stop the pump 104.

In various embodiments, valve 132 may then be closed and valve 134 may be opened by the micro-controller 404 using the valve driver 406. Pump 104 may drive positive pressure and pressure sensor 193 may monitor for pressure spikes indicating blockages. Check valve 138 may permit flow in the direction toward 171 but prevents backflow. An auto-sampling program may also check the pressure sensor 193 to confirm filling is complete (e.g., as pressure levels will drop once all liquid has been dispensed).

Valve 132 may then be opened as valve 134 is closed, and the pump flushes all remaining liquid back into the bioreactor 102 to minimize residual liquid in autosampler. Sensor 150 may monitor progress of this step, and also pressure sensor 193 may detect pressure spikes indicating a blockage. Once this is complete, all valves 132, 134 may close and the pump 104 may be stopped. The microcontroller 404 may indicate to operator through HMI 304 that the operation is now complete. The process may now complete or may be repeated as needed to generate a larger sample or more samples.

FIG. 5 is a schematic diagram of a computer system 500 in accordance with various embodiments. The computer system 500, upon which embodiments of the present teachings may be implemented. In various embodiments of the present teachings, computer system 500 may include a bus 502 or other communication mechanism for communicating information, and a processor 504 coupled with bus 502 for processing information. In various embodiments, computer system 500 can also include a memory, which can be a random-access memory (RAM) 506 or other dynamic storage device, coupled to bus 502 for determining instructions to be executed by processor 504. Memory also can be used for storing temporary variables or other intermediate information during execution of instructions to be executed by processor 504. In various embodiments, computer system 500 can further include a read only memory (ROM) 508 or other static storage device coupled to bus 502 for storing static information and instructions for processor 504. A data store 510, such as a magnetic disk, optical disk, solid state drive, or spinning disk drive may be provided and coupled to bus 502 for storing information and instructions.

In various embodiments, computer system 500 can be coupled via bus 502 to a display 516, such as a cathode ray tube (CRT), liquid crystal display (LCD), or light emitting diode display (LED) for displaying information to a computer user.

In various embodiments, the display may comprise one or more light indicators. In various embodiments, a light indicator may be designed to convey a binary state (e.g., on or off). In various embodiments, a light indicator may be designed to convey multiple states. In various embodiments, one of many multiple states may be signaled by selecting a set of characteristics of light (e.g., wavelength, frequency, and intensity). In various embodiments, alternating between one set of characteristics to the next may allow conveyance of any message useful or helpful (e.g., operational running parameters, one or more specified errors, etc.).

An input device 512, including alphanumeric and/or other keys, a touch-screen interface, optical sensing device, physical or presence sensing device may be coupled to bus 502 for communicating information and command selections to processor 504. Another type of user input device 512 is a cursor control, such as a mouse, a trackball or cursor direction keys for communicating direction information and command selections to processor 504 and for controlling cursor movement on display 516. The input device 512 typically has two degrees of freedom in two axes, a first axis (i.e., x) and a second axis (i.e., y), that allows the device to specify positions in a plane. However, it should be understood that input devices 512 allowing for 3-dimensional (x, y and z) cursor movement are also contemplated herein.

In various embodiments, computer system 500 can be coupled via bus 502 to one or more data ports 514. In various embodiments, the one or more data ports 514 may enable electronic communication between the components via bus 502 of the computer system 500 and/or the components 302, 304, 306, 150, 152, 132, 134, 193, 104 of a system for aseptically sampling a fluid 100 in accordance with various embodiments.

Consistent with certain implementations of the present teachings, results can be provided by computer system 500 in response to processor 504 executing one or more sequences of one or more instructions contained in memory 506. Such instructions can be read into memory 506 from another computer-readable medium or computer-readable storage medium. Execution of the sequences of instructions contained in memory 506 can cause processor 504 to perform the processes described herein. Alternatively, hard-wired circuitry can be used in place of or in combination with software instructions to implement the present teachings. Thus, implementations of the present teachings are not limited to any specific combination of hardware circuitry and software.

According to various embodiments, computer-readable medium (e.g., data store, data storage, etc.) or computer-readable storage medium may comprise any media that participates in providing instructions to processor 504 for execution. Such a medium can take many forms, including but not limited to, non-volatile media, volatile media, and transmission media. Non-limiting examples of non-volatile media can include optical, solid state, and magnetic disks, such as 508. Examples of volatile media can include, but are not limited to, dynamic memory, such as memory 506. Examples of transmission media can include, but are not limited to, coaxial cables, copper wire, and fiber optics, including the wires that comprise bus 502.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 504 of computer system 500 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc. In various embodiments, output devices 518 such as printers and displays may be used to present output files generated by the processes described herein.

In various embodiments, a computer system 500 may be used to coordinate sampling operations with other automated equipment. In various embodiments, a computer system 500 may be used to coordinate sampling with one or more of the systems described herein (e.g., an autosampler and an analyzer module).

### Exemplary Methods

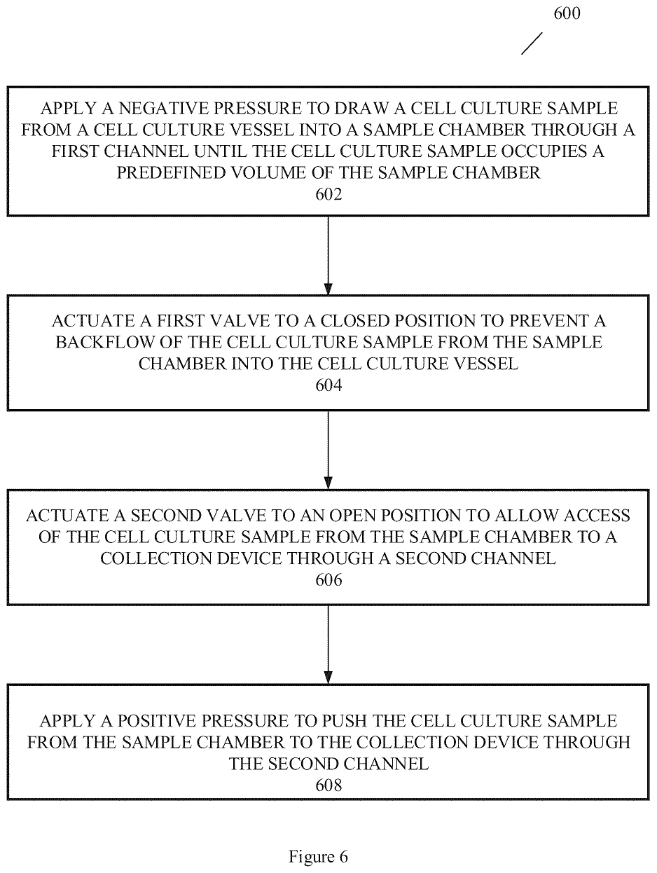

FIG. 6 is a method for aseptically sampling a fluid in accordance with various embodiments. In various embodiments, the methods described herein may be performed on the systems and devices for aseptically sampling a fluid as described in FIGS. 1, 2, 3, 4, and 5. For reference, non-limiting examples of part numbers may be enumerated in the methods description for a better understanding of how and when the methods may be performed.

Step 602 of the method comprises applying a negative pressure to draw a cell culture sample from a cell culture vessel into a sample chamber through a first channel until the cell culture sample occupies a predefined volume of the sample chamber according to various embodiments. For a non-limiting example, a cell culture sample may be drawn from a cell culture vessel 102 through a first end 161 of a first fluid channel 160 and pass through a second end 162 of the first fluid channel 160 where the first end 161 may be connected to the cell culture vessel 102 and the second end 162 may be connected a sample chamber 123 according to various embodiments. In various embodiments, the negative pressure may be applied by a pump 104 which may be fluidically coupled to the sample chamber 123 either directly or indirectly. For example, the pump 104 may apply force through a fourth fluid channel 190 and the fourth fluid channel 190 may be fluidically coupled to the pump 104 at a first end 191 of the fourth fluid channel 190 and a second end 192 of the fourth fluid channel may be fluidically coupled to a filter 140 according to various embodiments. Further, an opposing side of the filter 140 may be fluidically coupled to a third fluid channel 180 in various embodiments. In various embodiments, the third fluid channel 180 may comprise a first end 181 that may be fluidically coupled to the filter 140 and a second end 182 that may be fluidically coupled to the sample chamber 123. In various embodiments, a second sensor 152 may be positioned in such a way as to detect the presence of the cell culture sample within the third fluid channel 180. In various embodiments, a second sensor 152 may be positioned in such a way as to detect the presence of the cell culture sample within the sample chamber 123. In various embodiments, a first sensor 150 may detect the presence of the cell culture sample as it passes through the first fluid channel 160.

Step 604 of the method comprises actuating a first valve to a closed position to prevent a backflow of the cell culture sample from the sample chamber into the cell culture vessel according to various embodiments. For a non-limiting example, when the sample chamber 123 fills to its predefined volume a second sensor 152 may detect the presence of the cell culture sample according to various embodiments. When detection occurs, the first sensor 152 may send a signal to a controller 302 which, in turn may then send a signal to a first valve 132 to move into a closed position according to various embodiments. All signals described herein may comprise electronic communication via any means necessary and not limited to wired or wireless transmission according to various embodiments.

Step 606 of the method comprises actuating a second valve to an open position to allow access of the cell culture sample from the sample chamber to a collection device through a second channel according to various embodiments. In various embodiments, after the sample chamber 123 may have reached its predefined volume the controller 302 may instruct a second valve 134 to move from a closed position to an open position. In various embodiments, a controller 302 may determine when to actuate the second valve 134 based on data received from the second sensor 152.

Step 608 of the method comprises applying a positive pressure to push the cell culture sample from the sample chamber to the collection device through the second channel according to various embodiments. In various embodiments, the positive pressure may be applied by the pump 104 which may pump in an opposing direction to that when it applies a negative pressure. In various embodiments, the controller 302 may determine when to actuate the pump 104 in either the forward or reverse direction based on data received from one or more sensors 150, 152. In various embodiments, a control signal may be sent from the controller 302 to the pump 104 to apply the pressure.

In various embodiments, the method a purge step. In various embodiments, the purge step may include opening a first valve 132 and closing a second valve 134 and applying positive pressure until excess liquid in the fluidic assembly 200 is returned to a cell culture vessel 102.

In various embodiments, the method further comprises the step of activating a pump 104 in a forward direction to apply the positive pressure. In various embodiments, the method further comprises the step of activating the pump 104 in a reverse direction to apply the negative pressure. As described herein, control of the pump 104 may be monitored and adjusted based on sensor feedback received from one or more sensors 152, 154, 193 of the fluidic handling system 200.

In various embodiments, the method further comprises the step detecting the presence of the cell culture sample within the first channel using a first sensor. In various embodiments, the method may make use of a first sensor 150 in physical, sonic, or optical communication at a position between the first and the second ends 161, 162 of the first fluid channel 160, In various embodiments, the first sensor 150 may be for detecting the presence of a liquid (e.g., the cell culture sample). In various embodiments, the first sensor 150 may measure one or more of an optical characteristic, a capacitance, and/or an ultrasonic quality.

In various embodiments, the method may further comprise detecting the presence of the cell culture sample using a second sensor. In various embodiments, the method may make use of a second sensor 152 in physical, sonic or optical communication at a position between the first and the second ends 181, 182 of the third fluid channel 180. In various embodiments, the second sensor 152 may measure one or more of an optical characteristic, a capacitance, and/or an ultrasonic quality.

In various embodiments, the method may further comprise discontinuing the application of negative pressure once detection of the presence of the cell culture by the second sensor has occurred. In various embodiments, a controller 302 may send a signal to a pump 104 to discontinue application of negative pressure. In various embodiments, a controller 302 may send a signal to a pump 104 to initiate application of positive pressure.

In various embodiments, the method may comprise using the second sensor to detect the cell culture sample in the sample chamber. In various embodiments, the method may comprise using the second sensor to detect the cell culture sample in a third channel. In various embodiments, the third channel may be in fluid communication with the sample chamber.

In various embodiments, the sample chamber 123 may comprise a predefined volume. In various embodiments, the method may comprise filling the sample chamber 123 to a predefined volume. In various embodiments, the predefined volume may be based on a nature and quality of analytical testing once the method deposits the cell culture sample into a collection device 106.

In various embodiments, the sample chamber 123 may be fluidically coupled to the second end 162 of the first fluid channel 160 at a first position. In various embodiments, the sample chamber 123 may be fluidically coupled to the second end 172 of the second fluid channel 170 at a second position. In various embodiments, the sample chamber 123 may be fluidically coupled to the second end 182 of the third fluid channel at a third position.

In various embodiments, the sample chamber 123 may comprise a rectangular shape. In various embodiments, the first position and the second position may be located on opposing surfaces of the sample chamber 123. In various embodiments, the first, second, and third positions may be located on different walls of a predefined shape of the sample chamber 123 (e.g., shapes having three or more sides). In various embodiments, the first and second positions of the sample chamber 123 may be located at a based or bottom position of the sample chamber 123 and be opposed to one another while the third position may be located perpendicular (e.g., a top or upper location) to the first and second positions.

In various embodiments, the method may further comprise preventing the cell culture fluid from entering a fourth channel from the third channel using a filter, wherein the fourth channel connects the pump to the third channel. In various embodiments, the method may comprise using a fourth channel 190 having a first end 191 and a second end 192 to apply a pressurized fluid to the system. In various embodiments, a filter 140 may be affixed to the second end 192 of the fourth channel 190. In various embodiments, the filter 140 may comprise a luer lock for securing the second end 192 of the fourth fluid channel 190 to the first end 181 of the third fluid channel 180.

In various embodiments, the method may comprise preventing backflow from the collection device to the sample chamber using a check valve. In various embodiments, the method may comprise the step of keeping the system for aseptically sampling a cell culture fluid sterile by using the check valve 138 to prevent backflow. In various embodiments, the check valve 138 may be positioned along the second channel.

In various embodiments, the method may comprise mounting a fluidic assembly 200 to a fluidic handling system 300. In various embodiments, the fluidic assembly 200 comprises first, second, and third channels 160, 170, 180 a filter 140, and a check valve 138. In various embodiments, the fluidic handling system comprises a first and second sensors 150, 152 the first and second valves 132, 134 and the pump 104. In various the mounting step comprises welding a first end 161 of the first channel 160 to the cell culture vessel 102. In various embodiments, the mounting step comprises using an aseptic connector to connect the first end 161 of the first channel 160 to the cell culture vessel 102. In various embodiments, the mounting step may include securing one or more of the channels 160, 170, 180, 190 to fasteners of the fluidic handling system 300.

In various embodiments, the method may comprise performing an integrity test of the fluidic assembly 200 by activating the pump to apply pressurized air to the fluidic assembly 200 and checking for leaks prior to drawing the cell culture sample.

In various embodiments, an integrity test may comprise of the following steps:
1. All pinch valves 132, 134 closed.
2. Pump 104 begins to deliver positive pressure until pressure sensor 193 detects a threshold pressure which signals for the pump 104 to stop.
3. Pressure value (P_initial) may be recorded and a timer may begin for a predetermined duration (e.g., one minute).
4. Once timer has expired, pressure value (P_final) may be again recorded.
5. A pressure drop may be calculated by (P_inital-P_final). If pressure drop is below a predefined threshold, then integrity test is passed, otherwise it has failed.

In various embodiments, an extended version of a pressure integrity test may also be performed with pinch valve 132 open in order to include the bioreactor connection. Steps 2-4 may be the same, but with different threshold/times. In various embodiments, times and thresholds may be determined empirically for an application of interest.

In various embodiments, the method may comprise collecting data from the first and second sensors 150, 152 using a control system. In various embodiments the collected data may comprise usage and/or error logs, and sensor values. In various embodiments, the collected data may be recorded on onboard storage 510 and/or accessed through an external interface.

In various embodiments, the method may comprise actuating the first and second valves 132, 134 based on the data received by the control system and activating the pump 104 in one of a forward or reverse direction based on the data and the position of the first and second valves 132, 134.

In various embodiments, the method may comprise analyzing the cell culture sample for a quality. In various embodiments, the quality may include a cell count. In various embodiments, the quality may include contaminant information. In various embodiments, the quality may include cell viability information for one or more populations of cells within the cell culture sample. In various embodiments, the quality may include morphology information for one or more populations of cells within the cell culture sample, In various embodiments, the quality may include protein expression information for one or more populations of cells within the cell culture sample. In various embodiments, the protein expression information may include cell surface protein expression. In various embodiments, the method may employ chemical analysis methods. In various embodiments, a chemical analysis method may comprise metabolite analysis. In various embodiments, a chemical analysis method may comprise nutrient analysis. In various embodiments, a chemical analysis method may comprise pH analysis. In various embodiments, a chemical analysis method may comprise dissolved gas analysis.

In various embodiments, the method for sampling the cell culture fluid further comprises interrogating a fluid within the collection device for a quality of the fluid. In various embodiments, the quality includes at least one of a cell count, a contaminant, cell viability information for one or more populations of cells within the cell culture sample, morphology information for one or more populations of cells within the cell culture sample, and protein expression information for one or more populations of cells within the cell culture sample.

In various embodiments, the method further comprises sensing the presence of a fluid within the collection device.

### Equivalence

Those skilled in the art will recognize or be able to ascertain using no more than routine experimentation, many equivalents to the specified embodiments of the technologies described herein. It is to be understood that the technologies encompass all variants, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the technologies can be explicitly excluded from the claims, regardless of whether the specific exclusion is recited in the specification. The scope of the present disclosure is not limited to the description herein, but rather is as set forth in the claims below.

1. A system for sampling a cell culture fluid, comprising:
a fluidic assembly, comprising:
a first fluid channel having a first end and a second end;
a second fluid channel having a first end and a second end;
a third fluid channel having a first end and a second end; and
a sample chamber having a predefined volume, wherein the sample chamber is fluidically coupled to the second end of the first fluid channel at a first position, the second end of the second fluid channel at a second position, and the second end of the third fluid channel at a third position;
wherein the fluidic assembly is mounted to a fluidic handling system, the fluidic handling system comprising:
a first valve in physical communication between the first and second ends of the first fluid channel; and
a second valve in physical communication between the first and second ends of the second fluid channel.

2. The system for sampling the cell culture fluid of claim 1, wherein the fluidic assembly further comprises:
a check valve positioned between the first end and the second end of the second fluid channel; and
a filter affixed to the first end of the third fluid channel.

3. The system for sampling the cell culture fluid of claim 2, wherein the fluidic handling system further comprises:
a first sensor in physical, sonic, or optical communication at a position between the first and the second ends of the first fluid channel, wherein the first sensor is for detecting the presence of a liquid; and
a second sensor in physical, sonic, or optical communication at a position between the first and the second ends of the third fluid channel, wherein the second sensor is for detecting the presence of a liquid.

4. The system for sampling the cell culture fluid of claim 3, further comprising:
a fourth channel having a first end and a second end, wherein the filter is affixed to the second end of the fourth channel; and
a pressure sensor located between the first and seconds ends of the fourth channel and in fluidic communication with the fourth channel.

5. The system for sampling the cell culture fluid of claim 4, wherein the filter comprises a luer lock for securing the second end of the fourth fluid channel to the first end of the third fluid channel.

6. The system for sampling the cell culture fluid of claim 4, wherein a connector channel fluidically connects the pressure sensor to the fourth fluid channel.

7. The system for sampling the cell culture fluid of claim 1, wherein the fluid channels are each comprised of a flexible tube surrounding a hollow interior.

8. The system for sampling the cell culture fluid of claim 1, wherein the first and second valves each comprise a pinch valve.

9. The system for sampling the cell culture fluid of claim 1, further comprising a pump fluidically coupled to the first end of the fourth channel.

10. The system for sampling the cell culture fluid of claim 9, wherein the pump is selected from any one of a syringe pump, a diaphragm pump, or a peristaltic pump, or any pump capable of moving and compressing air.

11. The system for sampling the cell culture fluid of claim 1, further comprising a cell culture vessel fluidically coupled to the first end of the first fluid channel.

12. The system for sampling the cell culture fluid of claim 1, further comprising a collection device fluidically coupled to the first end of the second fluid channel.

13. The system for sampling the cell culture fluid of claim 12, further comprising a collection device sensor for detecting the presence of the collection device.

14. The system for sampling the cell culture fluid of claim 13, wherein the collection device sensor comprises a light emitting element, wherein the light emitting element operates at a first energy level to detect the presence of the collection device and a second energy level to interrogate a fluid within the collection device.

15. The system for sampling the cell culture of claim 12, further comprising a sensor for interrogating a fluid within the collection device.

16. The system for sampling the cell culture of claim 15, wherein the sensor physically, sonically, or optically interacts with the fluid.

17. The system for sampling the cell culture fluid of claim 12, wherein the collection device comprises an analytical device.

18. The system for sampling the cell culture fluid of claim 17, wherein the analytical device is selected from any one of a cell counter, a flow cytometer, and a cell imaging system.

19. The system for sampling the cell culture fluid according to claim 9, further comprising:
a controller, wherein the controller is in electronic communication with the pump, the pressure sensor, the first and second sensors, and the first and second valves; and
a human machine interface in electronic communication with the control unit.

20. (canceled)

21. (canceled)

22. A method for sampling a cell culture fluid, comprising:
applying a negative pressure to draw a cell culture sample from a cell culture vessel into a sample chamber through a first channel until the cell culture sample occupies a predefined volume of the sample chamber;
actuating a first valve to a closed position to prevent a backflow of the cell culture sample from the sample chamber into the cell culture vessel;
actuating a second valve to an open position to allow access of the cell culture sample from the sample chamber to a collection device through a second channel; and
applying a positive pressure to push the cell culture sample from the sample chamber to the collection device through the second channel.

23-44. (canceled)
